(19) United States
(12) Patent Application Publication
(10) Pub. No.: US 2011/0250678 A1
(43) Pub. Date: Oct. 13, 2011

(54) SYSTEM FOR PRODUCING GAS FROM ORGANIC WASTE

(76) Inventor: Sung-Ho JOO, Seoul (KR)

(21) Appl. No.: 13/077,018

(22) Filed: Mar. 31, 2011

(30) Foreign Application Priority Data
Apr. 8, 2010 (AU) 2010-201398

(51) Int. Cl. C12M 1/107; C12M 1/00

(52) U.S. Cl. 435/300.1; 435/289.1

(57) ABSTRACT

A system for producing gas from organic waste includes a pretreatment unit, which crushes organic waste; an oxidation unit, which oxidizes the crushed organic waste in an oxidation bath; an oxidation unit, which converts the oxidized organic waste into fluid by anaerobic digestion; and a stabilization unit, which divides the fluid into gas and liquid fertilizer when the fluid is supplied into a fluid-stabilizing bath. The oxidation bath has a transparent safety window, a coupler, a first pipe coupled to the coupler and connected to the oxidation pump, a second pipe, a pit formed in a bottom of the oxidation bath. An inorganic-material-discharging unit has one side seated in the pit and the other side exposed from above. A mixer is coupled to a lower inside wall of the oxidation bath corresponding to the pit, and includes first blades coupled to a mixing motor.

### CROSS-REFERENCE TO RELATED APPLICATIONS

The present application claims priority under 35 U.S.C. 119 of Australian Patent Application No. 2010-201398, filed Apr. 8, 2010, the disclosure of which is expressly incorporated by reference herein in its entirety.

### BACKGROUND OF THE INVENTION

1. Field of the Invention

The present invention relates to a system for producing gas from organic waste, and more particularly, to one which, through a set of processes of crushing the organic waste, such as food and drink leftovers, excrement, and kitchen scraps, into fine particles using a pretreatment unit, temporarily storing the crushed organic waste in a reservoir, and carrying the crushed organic waste through an oxidation unit and a digestion unit, decomposes the organic waste into gas, composed of methane gas and carbon dioxide, and liquid fertilizer by oxidation and anaerobic digestion, stabilizes and traps the decomposed methane gas, supplies the methane gas into a combustion unit to burn, and heats water or generates electricity using heat generated by the combustion of the methane gas, thereby making it easy to treat the organic waste and, as a result, reduce environmental pollution.

2. Description of Related Art

Due to industrial development, large cities contaminate the surrounding environment and cause imbalance in the ecosystem since they discharge a massive amount of waste that exceeds the cleaning capacity of nature. Of such waste, organic waste is mainly discharged from areas where population and plants are concentrated, in the form of sludge and excrement, and from food plants.

The organic waste, such as food and drink leftovers, city sewage sludge, and excrement, increases the pollution of soil and water when discharged in the form of liquid from its sources. If the organic waste is solid, it is collected from its sources and carried to a landfill. However, leachate, produced during the compression of the solid organic waste, causes incidental problems such as pollution of subterranean water and production of gas.

Excrement of livestock and waste of agriculture and fisheries are discharged from farming and fishing communities. However, these communities are not provided with cleaning facilities, which can separately treat the waste, or do not operate the cleaning facilities due to the burden of energy cost. Therefore, the waste has to be discharged through a cleaning process such as an activated sludge process.

Another approach converts part of organic waste into compost by inputting a certain amount of moisture controller into the organic waste to adjust the moisture content and blowing air into a fermenter to enable aerobic fermentation. However, this is not popular, since it is difficult to supply the moisture controller or ensure large fermenting-drying areas, and facilities are expensive.

The information disclosed in this Background of the Invention section is only for the enhancement of understanding of the background of the invention and should not be taken as an acknowledgment or any form of suggestion that this information forms a prior art that would already be known to a person skilled in the art.

### BRIEF SUMMARY OF THE INVENTION

Various aspects of the present invention provide a system for producing gas from organic waste, which, through a set of processes of crushing the organic waste, such as food and drink leftovers, excrement, and kitchen scraps, into fine particles and carrying the crushed organic waste through an oxidation unit and a digestion unit, decomposes the organic waste into gas, composed of methane gas and carbon dioxide, and liquid fertilizer by oxidation and anaerobic digestion, stabilizes and traps the decomposed methane gas, supplies the methane gas into a combustion unit to burn, and heats water or generates electricity using heat generated by the combustion of the methane gas, thereby making it easy to treat the organic waste and, as a result, reduce environmental pollution.

In an aspect of the present invention, the system for producing gas from organic waste may include a pretreatment unit, which discharges organic waste through a hopper, inputs the organic waste on a conveyor belt to a crusher, crushes the organic waste using the crusher, and stores the crushed organic waste in a reservoir; an oxidation unit, which carries the crushed organic waste into an oxidation bath using an oxidation pump and oxidizes the crushed organic waste in the oxidation bath; an oxidation unit, which carries the oxidized organic waste into a digestion bath and converts the oxidized organic waste into fluid, which includes gas and liquid fertilizer, by anaerobic digestion; and a stabilization unit, which divides the fluid into gas and liquid fertilizer when the fluid is supplied into a fluid-stabilizing bath. The oxidation bath can have a safety window provided in a sealed upper portion, the safety window made of transparent glass, a coupler provided in one side of the upper portion, a first pipe coupled to the coupler and connected to the oxidation pump, a second pipe penetrating through the other side of the upper portion, and a pit formed in a bottom of the oxidation bath. One side of an inorganic-material-discharging unit can be seated in the pit and the other side of the inorganic-material-discharging unit can be exposed from above the oxidation bath. A mixer can be coupled to a lower inside wall of the oxidation bath corresponding to the pit. The mixer can include a mixing motor and first blades coupled to the mixing motor.

According to exemplary embodiments of the present invention as set forth above, through a set of processes of crushing the organic waste, such as food and drink leftovers, excrement, and kitchen scraps, into fine particles and carrying the crushed organic waste through an oxidation unit and a digestion unit, the organic waste can be decomposed into gas, composed of methane gas and carbon dioxide, and liquid fertilizer by oxidation and anaerobic digestion, the decomposed methane gas can be stabilized, trapped, and supplied into a combustion unit to burn. As a result, it is possible to heat water or generate electricity using heat generated by the combustion of the methane gas.

Furthermore, it is possible to reduce environmental pollution by reducing the amount of organic waste, which would otherwise be discarded and buried.

The methods and apparatuses of the present invention have other features and advantages which will be apparent from, or are set forth in more detail in the accompanying drawings, which are incorporated herein, and in the following Detailed Description of the Invention, which together serve to explain certain principles of the present invention.

### BRIEF DESCRIPTION OF THE DRAWINGS

FIG. 1 is an illustration of a system for producing gas from organic waste according to an exemplary embodiment of the invention;

FIG. 2 is an illustration of an oxidation unit in the system for producing gas from organic waste according to an exemplary embodiment of the invention;

FIG. 3 is an enlarged view of a digestion unit in the system for producing gas from organic waste according to an exemplary embodiment of the invention;

FIG. 4 is an enlarged view of a stabilization unit in the system for producing gas from organic waste according to an exemplary embodiment of the invention; and

FIG. 5 is an enlarged view of a deodorizing stabilization unit in the system for producing gas from organic waste according to an exemplary embodiment of the invention.

### DETAILED DESCRIPTION OF THE INVENTION

Reference will now be made in detail to various embodiments of the present invention(s), examples of which are illustrated in the accompanying drawings and described below. While the invention(s) will be described in conjunction with exemplary embodiments, it will be understood that the present description is not intended to limit the invention(s) to those exemplary embodiments. On the contrary, the invention(s) is/are intended to cover not only the exemplary embodiments, but also various alternatives, modifications, equivalents and other embodiments that may be included within the spirit and scope of the invention as defined by the appended claims.

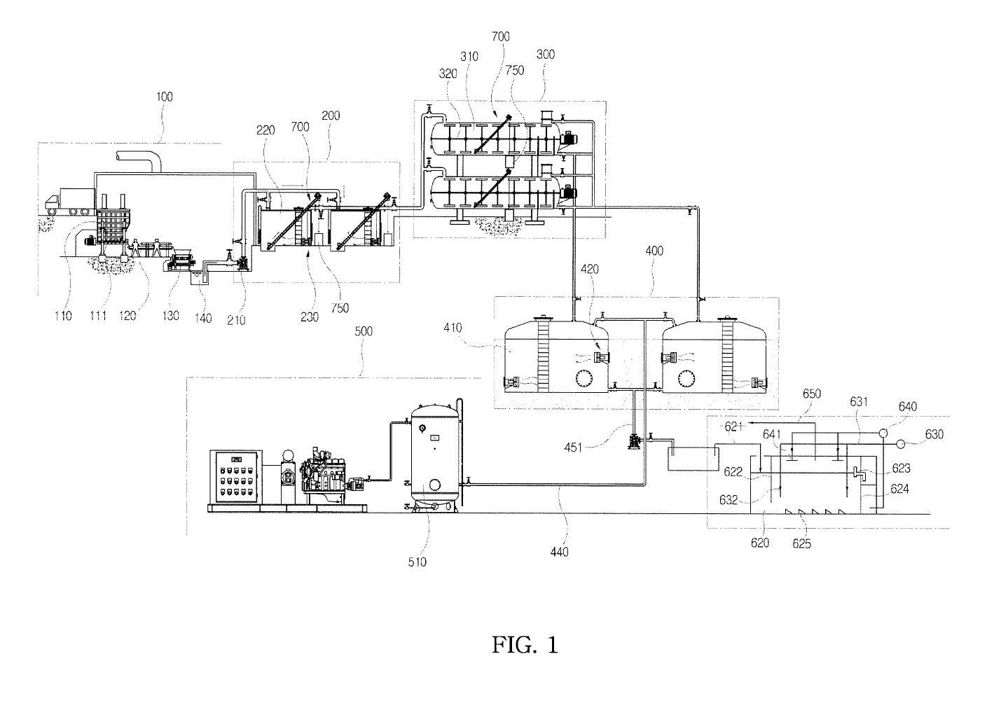

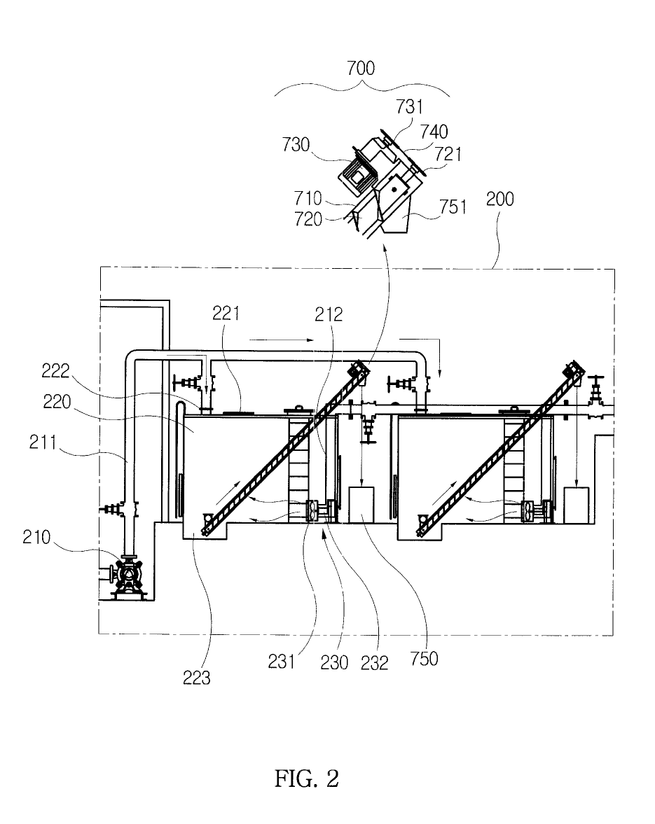

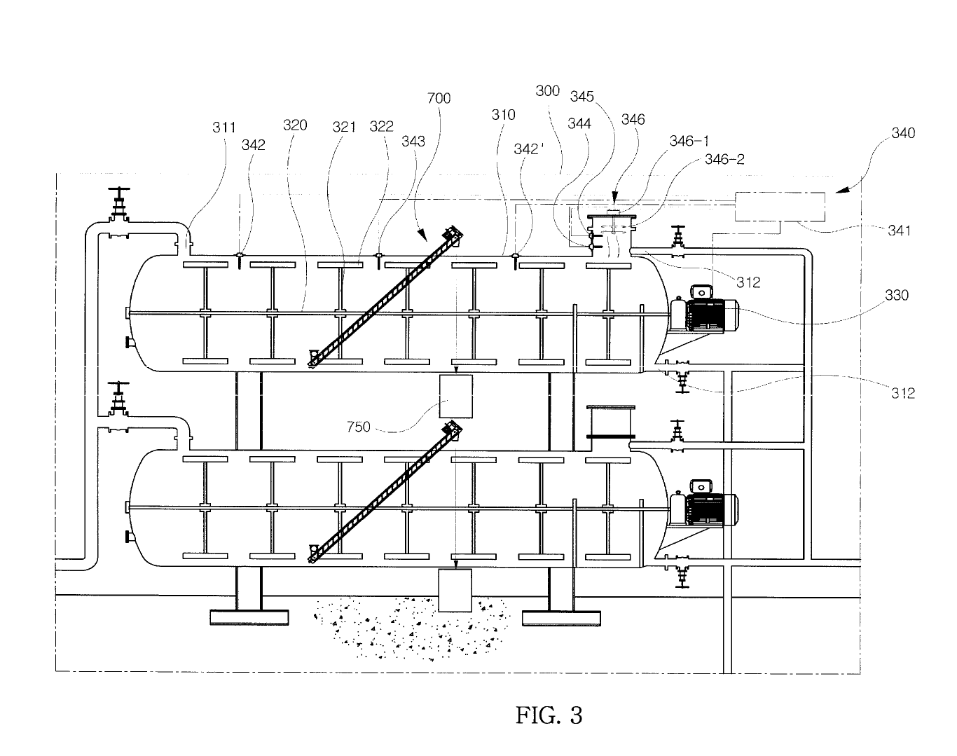

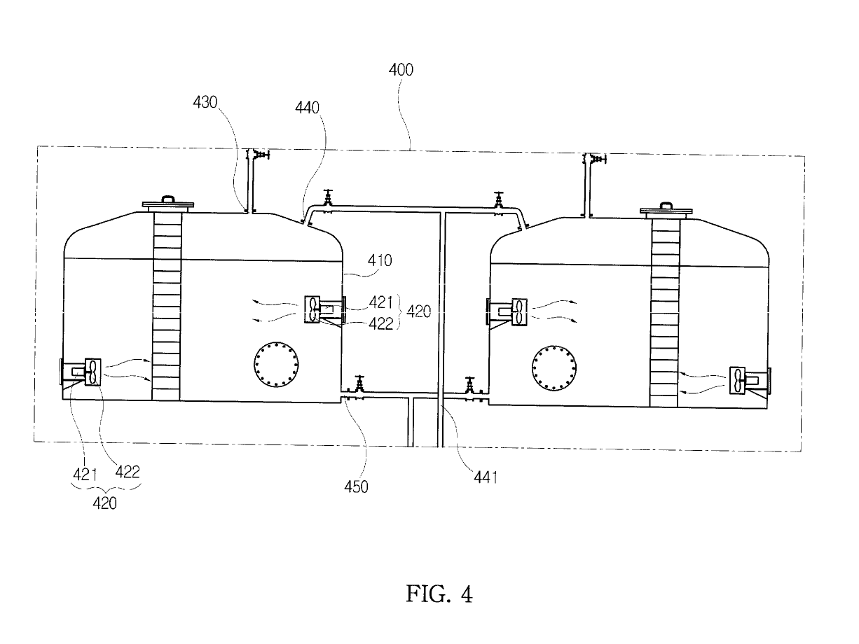

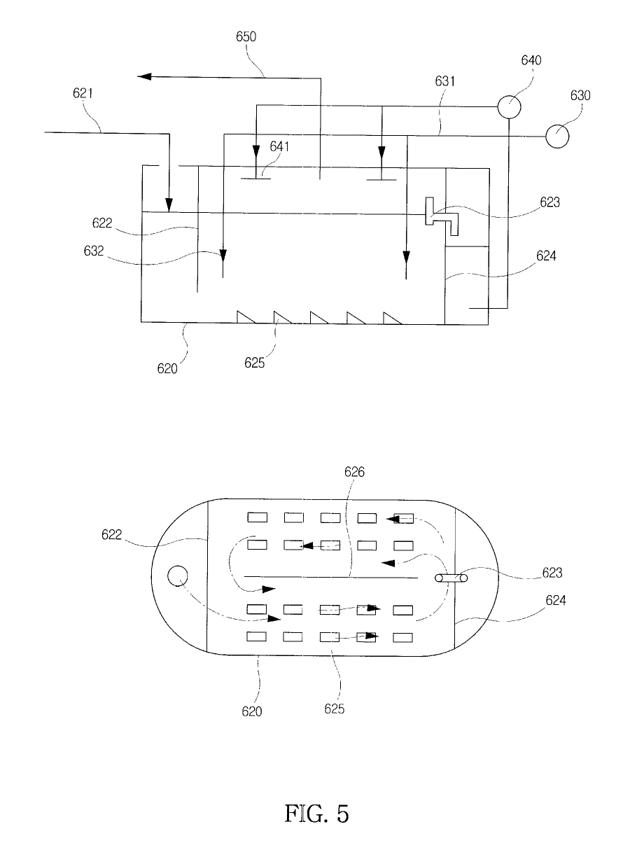

FIG. 1 is an illustration of a system for producing gas from organic waste according to an exemplary embodiment of the invention, FIG. 2 is an illustration of an oxidation unit in the system for producing gas from organic waste according to an exemplary embodiment of the invention, FIG. 3 is an enlarged view of a digestion unit in the system for producing gas from organic waste according to an exemplary embodiment of the invention, FIG. 4 is an enlarged view of a stabilization unit in the system for producing gas from organic waste according to an exemplary embodiment of the invention, and FIG. 5 is an enlarged view of a deodorizing stabilization unit in the system for producing gas from organic waste according to an exemplary embodiment of the invention.

As shown in FIGS. 1 to 5, the system for producing gas from organic waste includes a pretreatment unit 100, an oxidation unit 200, a digestion unit 300, and a stabilization unit 400. A combustion unit 500, which can burn methane gas, is connected to the stabilization unit 400, and a deodorizing stabilization unit 600 is connected to the stabilization unit 400 such that it can remove odors, which are created during the process of producing liquid fertilizer.

In the pretreatment unit 100, organic waste, which is carried in garbage trucks, is input into a hopper 110. The organic waste includes kitchen scraps, which are mainly discharged from food plants, food and drink leftovers, city sewage sludge, excrement, and the like. When the input organic waste is discharged at a constant speed by a transport screw 111, positioned below the hopper 110, it is carried on a conveyor belt 120 to a crusher 130, which then crushes the organic waste. Here, inorganic waste and impurities are separated manually by workers in advance from the organic waste on the conveyor belt 120.

The organic waste crushed by the crusher 130 is temporarily stored in a reservoir 140, which is buried below the crusher 130. Afterwards, under the pressure of an oxidation pump 210 of the oxidation unit 200, which is connected to the reservoir 140 by pipes, the organic waste is carried to an oxidation bath 220, in which the organic waste is oxidized.

When the crushed organic waste is carried to the oxidation bath 220, a mixing motor 232 rotates first blades 231 of a mixer 230, disposed on the inner wall in the lower portion of the oxidation chamber 220, thereby causing inorganic material in the crushed matter to be collected in a pit 223 in the bottom of the oxidation bath 220 while circulating inside the oxidation bath 220.

The inorganic material collected in the pit 223 of the oxidation bath 220 is discharged out by an inorganic-material-discharging unit 700. A drive motor 730 of the inorganic-material-discharging unit 700 rotates a drive sprocket wheel 731, which in turn rotates a follower sprocket wheel 721 via a chain 740 and then a screw shaft 720 coupled to the follower sprocket wheel 721. As the screw shaft 720 rotates inside a screw tube 710, the inorganic material collected in the inorganic-material-discharging unit 700 is discharged through the upper portion of the screw tube 710. The inorganic material discharged through the upper portion of the screw tube 710 is then loaded into an inorganic material reservoir 750 through a discharge hopper 751, which is connected to the outer edge of the screw tube 710, below the upper end of the screw tube 710. The inorganic material collected in this way can be discarded.

The crushed organic waste, supplied by the oxidation pump 210 during this process, is supplied into the oxidation bath 220 via a coupler 222 connected to a first pipe 211. While the crushed organic waste is stored and circulated inside the oxidation bath 220 for three or four days, it can be oxidized and decomposed into smaller elements, up to pH 4 to 5. The oxidized organic waste is carried to a digestion bath 310 of the digestion unit 300 through a second pipe 212. Here, the pressing action inside the oxidation bath 220 can be observed from outside through a safety window 221 made of transparent glass, in the upper portion of the oxidation bath 220. In addition, because the oxidation bath 220 is completely sealed, when the pressure inside the oxidation bath 220 rises too high, the safety window 221 breaks to protect the oxidation bath 220 and equipment installed inside the oxidation bath 220.

When the oxidized organic waste is carried to the oxidation unit 300, fluid containing gas and liquid fertilizer is produced by anaerobic digestion, and is then carried to fluid-stabilizing baths 410 of the stabilization unit 400, which divides the fluid into gas and liquid fertilizer.

The digestion bath 310 is formed as a horizontal cylinder with an inlet 311 and an outlet 312, and a shaft 320 is coupled inside the digestion bath 310 such that it can be rotated by a shaft motor 330, which is disposed outside the digestion bath 310. A plurality of mixing bridges 321 are provided around the shaft 320. One end of each of the mixing bridges 321 is coupled to the shaft 320, and a paddle 322 is coupled to the other end of the each mixing bridge 321, adjacent to the inner surface of the digestion bath 310. When the shaft 320 inside the digestion bath 310 is rotated following the rotation of the shaft motor 330, the paddles 322 connected to the mixing bridges 321 mix the crushed organic waste while carrying it at a low speed from one end to the other. The mixing bridges 321 coupled to the shaft 320 form a spiral structure in order to transport the crushed organic waste along the inside of the digestion bath 310 by moving one pitch when the shaft 320 rotates once.

When the crushed organic waste is being agitated and transported inside the digestion bath 310 by the mixing bridges 321 and the paddles 322, the inorganic material in the organic waste is discharged out by an inorganic-material-discharging unit 700, which is provided in the digestion bath 310 like the inorganic-material-discharging unit 700 in the oxidation unit 200. After discharged from the digestion bath 310, the inorganic material is discarded. Here, the crushed organic waste produces gas and liquid fertilizer while being decomposed by anaerobic digestion. The gas is mainly composed of methane gas and carbon dioxide with a small amount of odor.

The shaft motor 330, which rotates the shaft 320 inside the digestion bath 310, can be variably operated by a separate variable regulator 340. With the variable regulator 340, which variably operates the shaft motor 330, it is possible to increase or decrease the degree of decomposing the crushed organic waste.

Specifically, pH sensors 342 and 342′ are connected to the input side of a controller 341 of the variable regulator 340, adjacent to the inlet and the outlet of the digestion bath 310, a temperature sensor 343 is connected between the pH sensors 342 and 342′, and a carbon dioxide sensor 344 and a methane gas sensor 345 are connected to the outlet side. As signals detected by respective sensors are applied to the controller 341, the shaft motor 330 is variably operated. In addition, a bubble remover 346 is provided in the outlet side of the oxidation bath 310. The bubble remover 346 has one or more fans, which are rotated by a motor 346-1 to remove bubbles.

Although the fans 346-2, which are rotated by the motor 346-1, are employed to remove bubbles in an exemplary embodiment of the invention, nozzles and a pump can be provided in place of the motor 346-1 and the fans 346-2. In this case, it is possible to remove bubbles, concentrated in the outlet side of the digestion bath 310, by spraying liquid fertilizer, decomposed inside the digestion bath 310, through the nozzles.

Fluid, i.e., gas and liquid produced inside the digestion bath 310 is supplied to the stabilization unit 400 along pipes. When supplied to the stabilization unit 400, the fluid is stabilized by being circulated by circulators 420 in the fluid-stabilizing baths 410. In a respective fluid-stabilizing bath 410, one circulator 420 is coupled to the upper portion in one side and the other circulator 420 is coupled to the lower portion in the other side of the fluid-stabilizing bath 410.

In this case, the fluid-stabilizing bath 410 is buried such that the upper portion is exposed from the ground. Each circulator 420 has second blades 422 coupled to a circulation motor 421 so that it can circulate the fluid in proportion to the speed of the rotation of the second blades 422.

A fluid inlet 430 is coupled to one side of the upper portion of the fluid-stabilizing bath 410 such that the fluid decomposed in the digestion unit 300 can be easily supplied into the fluid-stabilizing bath 410. A gas outlet 440 and a pressure regulator (not shown) are coupled to the other side of the upper portion of the fluid-stabilizing bath 410 such that methane gas can be stably supplied to a combustor 510 of the combustion unit 500 through a third pipe 441, which is coupled to and extends from the gas outlet 440, and then be burned in the combustor 510.

According to an exemplary embodiment of the invention, gas stabilized in the fluid-stabilizing bath 410 of the stabilization unit 400 is supplied to and burned in the combustion unit 500, whereas fluid in the fluid-stabilizing bath 410 is supplied to the deodorizing stabilization unit 600 through a fourth pipe 451 coupled to a separate fluid outlet 450.

Aerobic fermentation fungi and photosynthetic bacteria are supplied to the deodorizing stabilization unit 600. The deodorizing stabilization unit 600 includes a stabilizer tank 610, the temperature of which is maintained at 60 to 70° C., and a deodorizing bath 620, which is connected to the outlet side of the stabilizer tank 610.

Waste water is input from the stabilizer tank 610 through a waste water inlet duct 621, which is in a sealed upper portion of the deodorizing bath 620. Since an inlet partition wall 622 having an open lower end is coupled to the inside of the deodorizing bath 620, the waste water is supplied toward an outlet partition 624 through the lower portion of the deodorizing bath 620.

A plurality of mixing protrusions 625 is coupled to the bottom of the inside of the deodorizing bath 620 such that the waste water can be smoothly and efficiently mixed while being circulated in one direction by a circulation partition 626 coupled to a space between the inlet partition 622 and the outlet partition 624. A circulating force, which causes the waste water to circulate, is provided by the pressure of air having a high pressure and a high pressure, which is supplied by an air pump 630, an air duct 631, and air nozzles 632.

Here, water nozzles 641 are provided in a space, in which the circulation partition 626 is formed, and are connected to a water pump 640. Thus, bubbles, which are created in this process, can be reduced by water, which is injected through the water nozzles 641.

Ammonia-containing gas, produced in this process, is returned to the stabilizer tank 610 through an outlet pipe 650. One end of the outlet pipe 650 is coupled to the upper portion of the deodorizing bath 620, and the other end of the outlet pipe 650 is coupled to the inlet side of the stabilizer tank 610. Thus, a small amount of ammonia is removed while being continuously circulated.

When the circulation of the waste water is finished, water and bacteria are separated from each other, located respectively in upper and lower areas. Then, the water is discharged as a decanter 623, which is provided in the outlet partition 624, moves up and down depending on the water level.

The foregoing descriptions of specific exemplary embodiments of the present invention have been presented for the purposes of illustration and description. They are not intended to be exhaustive or to limit the invention to the precise forms disclosed, and obviously many modifications and variations are possible in light of the above teachings. The exemplary embodiments were chosen and described in order to explain certain principles of the invention and their practical application, to thereby enable others skilled in the art to make and utilize various exemplary embodiments of the present invention, as well as various alternatives and modifications thereof. It is intended that the scope of the invention be defined by the Claims appended hereto and their equivalents.

What is claimed is:

1. A system for producing gas from organic waste comprising:
a pretreatment unit, which discharges organic waste through a hopper, inputs the organic waste on a conveyor belt to a crusher, crushes the organic waste using the crusher, and stores the crushed organic waste in a reservoir;
an oxidation unit, which carries the crushed organic waste into an oxidation bath using an oxidation pump and oxidizes the crushed organic waste in the oxidation bath;
an oxidation unit, which carries the oxidized organic waste into a digestion bath and converts the oxidized organic waste into fluid, which includes gas and liquid fertilizer, by anaerobic digestion; and
a stabilization unit, which divides the fluid into gas and liquid fertilizer when the fluid is supplied into a fluid-stabilizing bath,
wherein the oxidation bath has a safety window provided in a sealed upper portion, the safety window made of transparent glass, a coupler provided in one side of the upper portion, a first pipe coupled to the coupler and connected to the oxidation pump, a second pipe penetrating through the other side of the upper portion, a pit formed in a bottom of the oxidation bath, wherein one side of an inorganic-material-discharging unit is seated in the pit and the other side of the inorganic-material-discharging unit is exposed from above the oxidation bath, and a mixer coupled to a lower inside wall of the oxidation bath corresponding to the pit, wherein the mixer includes a mixing motor and first blades coupled to the mixing motor.

2. The system according to claim 1, wherein the digestion bath is formed as a horizontal cylinder with an inlet and an outlet, a shaft is coupled to a shaft motor, which is operated in response to a signal from a controller, one side of a plurality of mixing bridges is coupled to the shaft, the other side of the mixing bridges is coupled to a paddle, adjacent to an inner surface of the oxidation bath, and an inorganic-material-discharging unit is provided inside the horizontal cylinder.

3. The system according to claim 2, wherein the inorganic-material-discharging unit includes:
a screw tube;
a screw shaft inserted into the screw tube;
a follower sprocket wheel coupled to an upper end of the screw shaft;
a drive motor having a drive sprocket wheel, the drive motor coupled to an upper outer edge of the screw tube;
a chain connecting the drive sprocket wheel and the follower sprocket wheel with each other; and
a discharge hopper coupled to an outer edge of the screw tube, below an upper end of the screw tube, the discharge hopper connected to an inorganic material reservoir.

4. The system according to claim 1, wherein the inorganic-material-discharging unit includes:
a screw tube;
a screw shaft inserted into the screw tube;
a follower sprocket wheel coupled to an upper end of the screw shaft;
a drive motor having a drive sprocket wheel, the drive motor coupled to an upper outer edge of the screw tube;
a chain connecting the drive sprocket wheel and the follower sprocket wheel with each other; and
a discharge hopper coupled to an outer edge of the screw tube, below an upper end of the screw tube, the discharge hopper connected to an inorganic material reservoir.

5. The system according to claim 1, wherein the fluid-stabilizing bath is buried such that an upper portion is exposed from a ground, circulators are disposed inside the fluid-stabilizing bath, wherein one of the circulators is disposed on an upper portion of one side of the fluid-stabilizing bath and the other one of the circulators is disposed on a lower portion of the other side of the fluid-stabilizing bath, wherein each of the circulators includes a circulation motor and second blades coupled to the circulation motor, and wherein a fluid inlet is coupled to one side of an upper portion of the fluid-stabilizing bath, a gas outlet is coupled to the other side of the upper portion of the fluid-stabilizing bath, and a fluid outlet is coupled to a lower portion of the fluid-stabilizing bath.

6. The system according to claim 5, further comprising:
a combustion unit having a combustor, wherein a third pipe is coupled to and extends from the gas outlet to supply methane gas to the combustor of the combustion unit; and
a deodorizing stabilization unit, wherein a fourth pipe extending from the fluid outlet is coupled to the deodorizing stabilization unit, wherein aerobic fermentation fungi and photosynthetic bacteria are supplied to the deodorizing stabilization unit,
wherein a deodorizing bath is connected to an outlet side of a stabilizer tank, which has a temperature ranging from 60 to 70° C., a waste water inlet duct is coupled to a sealed upper portion of the deodorizing bath, an inlet partition wall having an open lower end is coupled to one end of an inside of the deodorizing bath, an outlet partition having a decanter is coupled to the other end of the inside thereof, a circulation partition is coupled to a space between the inlet partition and the outlet partition, air nozzles are disposed on spaces on both sides of the circulation partition and are connected to an air pump by an air duct, water nozzles are disposed on an upper space where the circulation partition is formed and are connected to a water pump, one end of an inlet pipe is coupled to the upper space of the deodorizing bath where the water nozzles are disposed, and the other end of the inlet pipe is coupled to an inlet side of the stabilizer tank.
